(19) United States
(12) Patent Application Publication
(10) Pub. No.: US 2015/0210755 A1
(43) Pub. Date: Jul. 30, 2015

(54) ANTI-POLY-N-ACETYL GLUCOSAMINE (PNAG) MONOCLONAL ANTIBODY AND USES THEREOF FOR THE PREVENTION OR TREATMENT OF PNAG EXPRESSING BACTERIAL INFECTION

(71) Applicant: SANOFI, Paris (FR)

(72) Inventors: Astrid Rey, Paris (FR); Cathy Cantalloube, Paris (FR); Meng Li, Bridgewater, NJ (US); Lei Tang, Bridgewater, NJ (US); Daniel Vlock, Concord, MA (US)

(73) Assignee: SANOFI, Paris (FR)

(21) Appl. No.: 14/421,667

(22) PCT Filed: Aug. 22, 2013
(86) PCT No.: PCT/US2013/056124

§ 371 Date: Feb. 13, 2015

Related U.S. Application Data
(60) Provisional application No. 61/693,001, filed on Aug. 24, 2012.

(30) Foreign Application Priority Data
Jul. 9, 2013 (FR) 1356743

(51) Int. Cl. C07K 16/12; G01N 33/68; A61K 9/00

(52) U.S. Cl. CPC C07K 16/1271; A61K 9/0019; G01N 33/6854; C07K 2317/565; A61K 2039/505

(57) ABSTRACT

Provided are methods for the treatment or prevention of microbial infections (e.g., bacterial infection) in which the underlying pathology involves a PNAG-expressing microbe (e.g., PNAG-expressing bacteria). The methods of the invention generally involve administering to the subject an effective amount of an antibody that specifically binds to PNAG. Such methods are particularly useful for the treatment of nosocomial staphylococcus (e.g., S. epidermidis and S. aureus) infections.

### RELATED APPLICATIONS

This application claims priority to U.S. Provisional Application Ser. No. 61/693,001, filed Aug. 24, 2012, and French Application No. 1356743, filed Jul. 9, 2013, each of which is incorporated herein by reference in its entirety.

### BACKGROUND OF THE INVENTION

Staphylococcus aureus (S. aureus) is a major cause of life-threatening infections in the developed world. The majority of hospital acquired S. aureas infections are now resistant to most of the common antibiotics and are known as Methicillin-resistant S. aureas (MRSA). The incidence of MRSA infections has dramatically increased in the last decade, occurring in both community-acquired and hospital-acquired cases. It has become the most common pathogen causing nosocomial infections. In the decade of the 1990s the number of nosocomial MRSA infections tripled despite current infection control recommendations (Noskin et al., Arch Intern Med. 165:1756-61, 2005).

It is generally accepted that the incidence of MRSA is rapidly increasing and is grossly under-estimated. One recent study found that as many as 20% of the 4.4 million patients admitted to US Intensive Care Units become infected or colonized with MRSA. That same study noted that as many as 30% of those patients develop a second MRSA infections following discharge.

Current therapy for MRSA generally relies on the use of the antibiotic vancomycin. However, since the late 1990's there has been an increasing incidence of vancomycin-resistant S. aureus. Accordingly, there is a need in the art for alternative methods of treating or preventing S. aureus infections that do not rely on the use of antibiotics.

### SUMMARY OF THE INVENTION

The present invention provides methods for the treatment or prevention of microbial infections (e.g., bacterial infection) in which the underlying pathology involves a PNAG-expressing microbe (e.g., S. aureus). The methods of the invention generally involve administering to the subject an effective amount of an antibody that specifically binds to PNAG. Such methods are particularly useful for the treatment of nosocomial staphylococcus (e.g., S. epidermidis and S. aureus) infections.

Accordingly, in one aspect, the invention provides a method for treating or preventing a poly-N-acetyl glucosamine (PNAG)-expressing bacterial infection in a subject comprising administering to the subject a therapeutically effective amount of an anti-PNAG antibody, wherein the antibody is administered at a dose of about 0.5 to about 20 mg/kg.

In one embodiment, the antibody is administered at a dose of about 10, about 15, about 17 or about 20 mg/kg. In certain exemplary embodiments, the antibody is administered at a dose of about 0.8, 1.0, 4.3, 5.0, 8.6, 10.0, 12.9, or 17.2 mg/kg.

In another embodiment, the administered dose achieves a serum level of anti-PNAG antibody of at least 10 μg/ml.

In another embodiment, the opsonic activity of the subject's serum remains essentially constant for at least 50 days after administration of the antibody.

In certain embodiments, the antibody has a serum half-life of at least 25 days.

In certain embodiments, the antibody is administered as a single dose. In other embodiments, the antibody is administered in multiple doses.

In certain embodiments, the dosing and scheduling of at least one dose is based upon a determination of the antibody's serum half-life in the subject and/or the in vitro opsonic activity of the subject's serum against PNAG-expressing bacteria.

In certain embodiments, the antibody is administered by intravenous infusion. In one embodiment, the intravenous infusion is administered over about 30 to about 120 minutes. In certain embodiments, the intravenous infusion volume is about 100ml.

In certain embodiments, the subject has a PNAG-expressing bacterial infection. In other embodiments, the subject is at risk of developing a PNAG-expressing bacterial infection.

In one embodiment, the infection is a lung infection, joint infection, endocardial infection, skin infection, soft tissue infection, or septicemia.

In another embodiment, the antibody is administered before, after or during a medical procedure. In certain embodiments, the medical procedure is the installation of a surgical implant in the subject. In exemplary embodiments, the surgical implant is a stent, catheter, cannula, prosthesis, or pace-maker.

In certain embodiments, the subject is a human.

In certain embodiments, the PNAG-expressing bacterial infection comprises Staphylococcus. In one embodiment, the Staphylococcus is S. epidermidis or S. aureus. In an exemplary embodiment, the S. aureus is Methicillin-resistant S. aureus.

In certain embodiments, the methods of the invention comprise determining the effective serum titer of the administered antibody using an in vitro opsonophagocytosis assay.

In certain embodiments, the antibody is in a formulation comprising: 10.2 mg/ml of anti-PNAG antibody; 20 mM NaPO₄; and 150 mM NaCl, wherein the pH of the formulation is 6.5.

In certain embodiments, the antibody is a human antibody. In certain embodiments, the antibody comprises a heavy chain variable region comprising the HCDR1, HCDR2, and HCDR3 amino acid sequences set forth in SEQ ID NOs: 1, 2, and 3, respectively. In certain embodiments, the antibody comprises a light chain variable region comprising the LCDR1, LCDR2, and LCDR3 amino acid sequences set forth in SEQ ID NOs: 4, 5, and 6, respectively. In particular embodiments, the antibody comprises a heavy chain variable region comprising the HCDR1, HCDR2, and HCDR3 amino acid sequences set forth in SEQ ID NOs: 1, 2, and 3, respectively, and a light chain variable region comprising the LCDR1, LCDR2, and LCDR3 amino acid sequences set forth in SEQ ID NOs: 4, 5, and 6, respectively. In another embodiment, the antibody comprises a heavy chain variable region comprising the amino acid sequence set forth in SEQ ID NO: 7. In another embodiment, the antibody comprises a light chain variable region comprising the amino acid sequence set forth in SEQ ID NO: 8. In yet another embodiment, the antibody comprises a heavy chain variable region comprising the amino acid sequence set forth in SEQ ID NO: 7, and a light chain variable region comprising the amino acid sequence set forth in SEQ ID NO: 8.

### BRIEF DESCRIPTION OF THE DRAWINGS

FIG. 1 depicts the results of an in vivo mouse assay measuring the protective activity of F598 (SAR279356) against S. pneumoniae infection in the respiratory tract.

FIG. 2 depicts the results of an in vivo mouse assay measuring the protective activity of F598 against S. aureus skin infection.

FIG. 3 depicts the serum half-life of F598 in human subjects at various doses.

FIG. 4 depicts the activity profile of F598 in human serum at various doses, as measured using an in vitro opsonophagocytic assay.

FIG. 5 depicts the activity profile of F598 in human serum at various doses, as measured using an in vitro opsonophagocytic killing assay.

### DETAILED DESCRIPTION

The present invention provides methods for the treatment or prevention of microbial infections (e.g., bacterial infection) in which the underlying pathology involves a PNAG-PNAG)-expressing microbe (e.g., S. aureus). The methods of the invention generally involve administering to the subject an effective amount of an antibody that specifically binds to PNAG. Such methods are particularly useful for the treatment of nosocomial staphylococcus (e.g., S. epidermidis and S. aureus) infections.

### I. Definitions

In order that the present invention may be more readily understood, certain terms are first defined.

As used herein, the terms “poly-N-acetyl glucosamine” or “PNAG” refer to a polymer of N-acetyl glucosamine monomers linked via a beta 1-6 linkage. The terms also encompass partially or fully deacylated poly-N-acetyl glucosamine.

As used herein, the term “PNAG-expressing bacterial infection” refers to a microbial infection comprising a PNAG-expressing microbe (e.g., S. aureus).

As used herein, the term “nosocomial infection” refers to infection acquired in a hospital or from a medical procedure performed inside or outside of a hospital. Exemplary nocosomial infections include sepsis (bloodstream infection), surgical site infection, or hospital-acquired pneuomia.

As used herein, the term “preventing a nocosomial infection” refers to an inhibition or reduction in the severity of an infection.

As used herein, the term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated V_{H}) and a heavy chain constant region. The heavy chain constant region comprises three domains, C_{H}1, C_{H}2 and C_{H}3. Each light chain comprises a light chain variable region (abbreviated V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

As used herein, the term “antigen-binding portion” of an antibody include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as diabodies, triabodies, tetrabodies and minibodies, are also encompassed within the expression “antigen-binding portion.”

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

As used herein the term “framework (FR) amino acid residues” refers to those amino acids in the framework region of an Ig chain. The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs.

As used herein, the term “specifically binds to” refers to the ability of an antibody or an antigen-binding fragment thereof to bind to an antigen with an Kd of at least about 1×10⁻⁶ M, 1×10⁻⁷M, 1×10⁻⁸M, 1×10⁻⁹M, 1×10⁻¹⁰ M, 1×10⁻¹¹ M, 1×10⁻¹² M, or more, and/or bind to an antigen with an affinity that is at least two-fold greater than its affinity for a nonspecific antigen.

As used herein, the term “antigen” refers to the binding site or epitope recognized by an antibody or antigen binding portion thereof.

As used herein, the term “effective amount” refers to that amount of an antibody or an antigen binding portion thereof that binds PNAG, which is sufficient to effect treatment, prognosis or diagnosis of a PNAG-expressing bacterial infection, as described herein, when administered to a subject. A therapeutically effective amount will vary depending upon the subject and disease condition being treated, the weight and age of the subject, the severity of the disease condition, the manner of administration and the like, which can readily be determined by one of ordinary skill in the art. Suitable dosages for administration are generally within the range of about 0.0001 to 1000 mg/kg, and more usually 0.1 to 100 mg/kg (e.g., about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20 mg/kg), of the host body weight. For example dosages can be within the range of about 0.5 mg/kg to about 20 mg/kg (e.g., from about 4.3 to about 12.9 mg/kg). Doses intermediate in the above ranges are also intended to be within the scope of the invention, e.g., about 0.9, 4.3, 8.6, 12.9 or 17.2 mg/kg. Dosage regiments may be adjusted to provide the optimum therapeutic response. An effective amount is also one in which any toxic or detrimental effects (i.e., side effects) of an antibody or antigen binding portion thereof are minimized and/or outweighed by the beneficial effects.

As used herein, the term “subject” includes any human or non-human animal.

### II. PNAG-Expressing Microbes

The methods of the invention can be used to treat or prevent a poly-N-acetyl glucosamine (PNAG)-expressing microbial (e.g., bacterial) infection in a subject. PNAG is expressed by a variety of microbes, including bacteria and fungi. In general, an infection caused by any PNAG-expressing microbe can be treated or prevented using the methods of the invention. PNAG-expressing bacteria amenable to treatment using the methods disclosed herein include, without limitation, Staphylococci (e.g., S. epidermis, S. aureus (e.g., Multi Drug Resistant S. aureus)), S. carnosus, S. haemolyticus, Pseudomonas aeruginosa, E. coli (e.g., E. coli 0157:H7 and E. coli CFT073), Yersinia pestis, Yersinia entercolitica, Xanthomonas axonopodis, Pseudomonas fluorescens, Actinobacillus actinomycetemcomitans, Actinobacillus pleuropneumoniae, Ralstonia solanacearum, Bordetella pertussis, Bordetella parapertussis and Bordetella bronchiseptica. In certain exemplary embodiments, the PNAG-expressing microbe is S. aureus (e.g., Multi Drug Resistant S. aureus)),

### III. Nosocomial Infections

The invention provides methods for treating or preventing nosocomial PNAG-expressing bacterial infection in a subject by administering to the subject an effective amount of an antibody that specifically binds to PNAG.

Any medical procedure, whether performed inside or outside of a hospital, that confers a risk to a patient of developing a PNAG-expressing bacterial infection can be treated or prevented using the methods of the invention. Examples of such medical procedures include, without limitation, surgery and implantation of a surgical device (e.g., catheter, cannula, prosthesis, respirator, mechanical ventilator, replacement heart valve, and pace-maker). Exemplary surgical devices include, without limitation: central venous catheters; peritoneal dialysis catheters; orthopedic prostheses; orthopedic mesh; intracardiac devices such as artificial valves, pacemakers, and stents; cochlear implants; breast implants; endotracheal tubes; voice prostheses; and intraocular lenses.

The skilled artisan will appreciate that in the case of an immunosupressed subject, the mere fact that the subject is in a hospital (or another environment that can harbor a PNAG-expressing microbe) identifies this subject as being at risk of developing a PNAG-expressing bacterial infection (e.g., in the lung, urinary tract or in an open wound) even if they do not receive a surgical procedure.

### IV. Anti-PNAG Antibodies

Any antibody that binds to PNAG and inhibits formation of a PNAG-expressing bacterial infection can be used in the methods of the invention. Exemplary antibody VH, VL and CDR amino acid sequences suitable for use in the invention are set forth in Table 1.

TABLE 1
VH, VL and CDR amino acid sequences
of exemplary anti-PNAG antibodies.

| | | SEQ |
|---|---|---|
| Antibody | Sequence | ID NO. |
| F598 HCDR3 | DTYYYDSGDYEDAFDI | 1 |
| F598 HCDR2 | YIHYSRSTNSNPALKS | 2 |
| F598 HCDR1 | GYYWS | 3 |
| F598 LCDR3 | QTWGAGIRV | 4 |
| F598 LCDR2 | VNRDGSHIRGD | 5 |
| F598 LCDR1 | TLSSGHSNYAIA | 6 |
| F598 VH | QVQLQESGPGLVKPSETLSLTCTVSGGSISGYY | 7 |
| | WSWIRQPPGKGLEWIGYIHYSRSTNSNPALKSR | |
| | VTISSDTSKNQLSLRLSSVTAADTAVYYCARDT | |
| | YYYDSGDYEDAFDIWGQGTMVTVSS | |
| F598 VL | QLVLTQSPSASASLGASVKLTCTLSSGHSNYAI | 8 |
| | AWHQQQPGKGPRYLMKVNRDGSHIRGDGIPDRF | |
| | SGSTSGAERYLTISSLQSEDEADYYCQTWGAGI | |
| | RVFGGGTKLTVLG | |
| F628 HCDR3 | DTYYESSGHWFDGLDV | 9 |
| F628 HCDR2 | YIHYSGSTNSNPSLKS | 10 |
| F628HCDR1 | NYYWS | 11 |
| F628LCDR3 | QTWGPGIRV | 12 |
| F628 LCDR2 | VKSDGSHSKGD | 13 |
| F628 LCDR1 | TLDSEHSRYTIA | 14 |
| F628VH | QVQLQESGPGLVKPSETLSLTCTVSGGSISNYY | 15 |
| | WSWIRQSPGRGLEWIGYIHYSGSTNSNPSLKSR | |
| | VTISVDTSKNQVSLKLGSVTAADTAIYYCARDT | |
| | YYESSGHWFDGLDVWGQGTSVTVSS | |
| F628 VL | QPVLTQSPSASASLGASVKLTCTLDSEHSRYTI | 16 |
| | AWHQQQPEKGPRYLMKVKSDGSHSKGDGITDRF | |
| | SGSSSGAERYLTISSLQSEDEADYYCQTWGPGI | |
| | RVFGGGTKLTVL | |
| F630 HCDR3 | DYYETSGYAYDDFAI | 17 |
| F630 HCDR2 | WVSTYNGRTNYAQKFRG | 18 |
| F630 HCDR1 | NFGIS | 19 |
| F630 LCDR3 | QTWGPGIRV | 20 |
| F630 LCDR2 | VNSDGSHTKGD | 21 |
| F630LCDR1 | TLSSGHSTYAIA | 22 |
| F630 VH | QVQLVQSGAEMKRPGASVKVSCKASGYTFTNFG | 23 |
| | ISWVRQAPGQGLEWIGWVSTYNGRTNYAQKFRG | |
| | RVTMTTDTSTNTAYMELRSLGSDDTAVFYCARD | |
| | YYETSGYAYDDFAIWGQGTLVTVSS | |
| F630 VL | QLVLTQSPSASASLGASVKLTCTLSSGHSTYAI | 24 |
| | AWHQQQPLRGPRFLMKVNSDGSHTKGDGIPDRF | |
| | SGSSSGAERYLSISSLQSEDESDYYCQTWGPGI | |
| | RVFGGGTKLTVLG | |

In certain embodiments, the antibody, or antigen binding fragment thereof, comprises one or more CDR region amino acid sequences selected from the group consisting of SEQ ID NO: 1, 2, 3, 4, 5, 6, 9, 10, 11, 12, 13, 14, 17, 18, 19, 20, 21, and 22.

In other embodiments, the antibody, or antigen binding fragment thereof, comprises HCDR3, HCDR2 and HCDR1 region amino acid sequences selected from the group consisting of:

a) SEQ ID NO: 1, 2 and 3;

b) SEQ ID NO: 9, 10 and 11; and

c) SEQ ID NO: 17, 18 and 19, respectively.

In other embodiments, the antibody, or antigen binding fragment thereof, comprises the LCDR3, LCDR2 and LCDR1 region amino acid sequences selected from the group consisting of:

a) SEQ ID NO: 4, 5 and 6;

b) SEQ ID NO: 12, 13 and 14; and

c) SEQ ID NO: 20, 21 and 22, respectively.

In other embodiments, the antibody, or antigen binding fragment thereof, comprises the HCDR3, HCDR2, HCDR1, LCDR3, LCDR2 and LCDR1 region amino acid sequences selected from the group consisting of:

a) SEQ ID NO: 1, 2, 3, 4, 5 and 6;

b) SEQ ID NO: 9, 10, 21, 12, 13 and 14; and

c) SEQ ID NO: 17, 18, 21, 20, 21 and 22, respectively

In other embodiments, the antibody, or antigen binding fragment thereof, comprises the VH region amino acid sequences set forth in SEQ ID NO: 7, 15 and/or 23.

In other embodiments, the antibody, or antigen binding fragment thereof, comprises the VL region amino acid sequences set forth in SEQ ID NO: 8, 16, and/or 24.

In other embodiments, the antibody, or antigen binding fragment thereof, comprises the VH and VL region amino acid sequences selected from the group consisting of: SEQ ID NO: 7 and 8; SEQ ID NO: 15 and 16; and SEQ ID NO: 23 and 24, respectively.

### V. Opsonophagocytic assays

In one aspect, the invention provides opsonophagocytic assays for measuring the functional titer of anti-PNAG antibodies in the serum of a subject. Such methods generally involve: obtaining serum from a patient; contacting the serum with phagocytic cells, complement, and PNAG-expressing bacteria; and measuring the amount of phagocytosis of the bacteria in the presence and absence of the serum. In certain embodiments, the subject has been administered an anti-PNAG antibody (e.g., the F598 antibody disclosed herein) prior to performance of the opsonophagocytic assay.

In certain embodiments, the amount of phagocytosis is measured by determining the number of bacteria killed during the assay. In other embodiments, the amount of phagocytosis is measured by determining the amount of bacteria phagocytosed during the assay. Such phagocytosis measurement can be achieved by, for example, fluorescently labeling the bacterial cells and determining uptake of labeled bacteria by the phagocytic cells using a Fluorescence Activated Cell Sorter (FACS).

Suitable phagocytic cells include, without limitation differentiated HL60. Bacteria suitable for use in the opsonophagocytic assays of the invention include, without limitation S. aureus strains MN8 or MN8m. For FACS assay, bacteria can be labeled with any FACS-detectable fluor including, without limitation, 5,6 Carboxyfluorescein succinimidyl ester. In certain embodiments, phagocytosis assays are performed using, 5,6 Carboxyfluorescein succinimidyl ester-labeled S. aureus.

### VI. Therapeutic Administration and Formulations

The invention provides methods for treating or preventing PNAG-expressing bacterial infection by administering to a subject in need of thereof a composition comprising an anti-PNAG antibody, or antigen binding fragment thereof. In accordance with the methods of the present invention, a therapeutically effective amount of an anti-PNAG antibody that is administered to a subject will vary depending upon, for example, the antagonist, type of infection, conditions, the age and the size (e.g., body weight or body surface area) of the subject, as well as the route of administration, and other factors well known to those of ordinary skill in the art.

In certain embodiments, the amount of the anti-PNAG antibody that is administered to a subject is expressed in terms of milligrams of antibody per kilogram of the subject's body weight (i.e., mg/kg). In one embodiment, the methods of the present invention include administering an anti-PNAG antibody to a subject at a dose of about 0.0001 to 1000 mg/kg, and more usually 0.1 to 100 mg/kg (e.g., about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20 mg/kg). For example, dosages can be within the range of about 0.5 mg/kg to about 20 mg/kg. In other embodiments, exemplary dosages can be within the range of about 10 to about 20 mg/kg, about 12 to about 20 mg/kg, about 13 to about 20 mg/kg, about 15 mg/kg to about 20 mg/kg, or about 18 mg/kg to about 20 mg/kg. Doses intermediate in the above ranges are also intended to be within the scope of the invention (e.g., about 1 (e.g., 0.9), about 5 (e.g., 4.3), about 10 (e.g., 8.6), about 15 (e.g., 12.9), or about 20 (e.g., 17.2) mg/kg).

In certain embodiments, the dose of anti-PNAG antibody administered to the subject is from about 0.1 to about 1000 mg per dose (e.g., about 1 to 100 mg, 100 to 200 mg, 200 to 300 mg, 300 to 400 mg, 500 to 600 mg, 600 to 700 mg, 700 to 800 mg, 800 to 900 mg, or 900 to 1000 mg).

The precise time of administration of the anti-PNAG antibody can be adjusted according to patient need. In the case of prophylactic treatment to inhibit the formation of a PNAG-expressing bacterial infection in a subject, the anti-PNAG antibody can be given at any time prior to exposure to the PNAG-expressing microbe (e,g., upon entry into hospital or at the start of a medical procedure). For example, the anti-PNAG antibody can be administered between 0 and 240 hours prior to exposure to the PNAG-expressing microbe, e.g., about 0.5, 1, 2, 3, 4, 5, 6, 7, 8, 9,10, 11, 12, 13, 14, 15, 16, 17, 18, 19, 20, 21, 22, 23 24, 30, 40, 60, 80, 90, 100, 110, 110, 120, 130, 140, 150, 160, 170, 180, 190, 200, 2010, 220, 230, and/or 240 hours.

In certain embodiments, the methods of the present invention include administering multiple doses of an anti-PNAG antibody to a subject over a specified time course. For example, the anti-PNAG antibody can be administered once, about once a week, about once every two weeks (q2w), or about once a month (q4w). In certain embodiments, the methods of the invention include administering a first dose of an anti-PNAG antibody to a subject at a first time point, followed by administering at least a second dose of an anti-PNAG antibody to the subject at a second time point. The first and second doses, in certain embodiments, may contain the same amount of anti-PNAG antibody. The time between the first and second doses may be from about a few hours to several weeks. For example, the second time point (i.e., the time when the second dose is administered) can be from about 1 hour to about 7 weeks after the first time point (i.e., the time when the first dose is administered). According to certain exemplary embodiments of the present invention, the second time point can be about 1 hour, about 4 hours, about 6 hours, about 8 hours, about 10 hours, about 12 hours, about 24 hours, about 2 days, about 3 days, about 4 days, about 5 days, about 6 days, about 7 days, about 2 weeks, about 4 weeks, about 6 weeks, about 8 weeks, about 10 weeks, about 12 weeks, about 14 weeks or longer after the first time point. In certain embodiments, the second time point is about 1 week or about 2 weeks. It is preferred that the second time point is about 2 weeks. Third and subsequent doses may be similarly administered throughout the course of treatment of the patient. It is preferred that the third dose is given about 2 weeks after the second dose, and that a fourth dose is given about 2 weeks after the third dose.

In some embodiments, the anti-PNAG antibody is administered by infusion. In some embodiments, the infusion is given over two hours. In some embodiments, the anti-PNAG antibody is administered to the subject under fasting conditions. In some embodiments, the anti-PNAG antibody is diluted in normal saline (0.9% sodium chloride) prior to infusion. In specific embodiments, the anti-PNAG antibody is diluted in 100 mL of normal saline.

In certain embodiment, the dosage of anti-PNAG antibody is adjusted to achieve a certain plasma antibody or toxin concentration, e.g., 1-1000 ug/ml. In certain embodiments, the plasma antibody concentration is greater than 10 ug/ml.

The invention provides methods of using therapeutic compositions comprising an anti-PNAG antibody. The therapeutic compositions of the invention will be administered with suitable carriers, excipients, and other agents that are incorporated into formulations to provide improved transfer, delivery, tolerance, and the like. In certain embodiments, the therapeutic composition comprises an anti-PNAG antibody in a buffer comprising 20 mM NaPO₄ and 150 mM NaCl, at pH 6.5. In a particular embodiment, the therapeutic composition comprises 10.2 mg/ml of anti-PNAG antibody.

A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., encapsulation in liposomes, microparticles, microcapsules, receptor mediated endocytosis (see, e.g., Wu et al. (1987) J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The Anti-PNAG antibody can be administered parenterally or subcutaneously.

The pharmaceutical composition can also be delivered in a vesicle, in particular a liposome (see Langer (1990) Science 249:1527-1533). In certain situations, the pharmaceutical composition can be delivered in a controlled release system, for example, with the use of a pump or polymeric materials. In another embodiment, a controlled release system can be placed in proximity of the composition's target, thus requiring only a fraction of the systemic dose.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

In accordance with the methods disclosed herein, the anti-PNAG antibody, or antigen binding fragment thereof, can be administered to the subject using any acceptable device or mechanism. For example, the administration can be accomplished using a syringe and needle or with a reusable pen and/or autoinjector delivery device. The methods of the present invention include the use of numerous reusable pen and/or autoinjector delivery devices to administer an anti-PNAG antibody (or pharmaceutical formulation comprising the antagonist). Examples of such devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park, Ill.), to name only a few.

The use of a microinfusor to deliver a therapeutic composition to a subject is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. No. 6,629,949; U.S. Pat. No. 6,659,982; and Meehan et al., J. Controlled Release 46:107-116 (1996). Microinfusors are particularly useful for the delivery of large doses of therapeutic proteins contained within high concentration (e.g., about 100, 125, 150, 175, 200 or more mg/mL) and/or viscous solutions.

### VII. Pharmaceutical Unit Dosage Form

In certain embodiments of the invention, the anti-PNAG antibody is packaged into a unit dosage form. The term “unit dosage form” as used in the specification and claims refers to physically discrete units suitable as unitary dosages for human and animal subjects, each unit containing a predetermined quantity of active material (e.g., about 10 to about 5000 mg of anti-PNAG antibody (e.g., about 102 mg of anti-PNAG antibody)) calculated to produce the desired therapeutic effect in association with the required pharmaceutical diluent, carrier or vehicle. The specifications for the novel unit dosage forms of this invention are dictated by and are directly dependent on (a) the unique characteristics of the active material and the particular therapeutic effect to be achieved, and (b) the limitation inherent in the art of compounding such an active material for therapeutic use in humans, as disclosed in this specification, these being features of the present invention. Examples of suitable unit dosage forms in accord with this invention are vials, tablets, capsules, troches, suppositories, powder packets, wafers, cachets, ampoules, segregated multiples of any of the foregoing, and other forms as herein described. The active ingredients to be employed as therapeutic agents can be easily prepared in such unit dosage form with the employment of pharmaceutical materials which themselves are available in the art and can be prepared by established procedures.

The following preparations are illustrative of the preparation of the unit dosage forms of the present invention, and not as a limitation thereof. Several dosage forms may be prepared embodying the present invention. For example, a unit dosage per vial may contain 1 ml, 2 ml, 3 ml, 4 ml, 5 ml, 6 ml, 7 ml, 8 ml, 9 ml, 10 ml, 15 ml, or 20 ml of anti-PNAG antibody or a fragment thereof ranging from about 10 to about 5000 mg of anti-PNAG antibody. In one embodiment, the dosage form comprises 102 mg of anti-PNAG antibody in 10 ml. If necessary, these preparations can be adjusted to a desired concentration by adding a sterile diluent to each vial. In one embodiment, the ingredients of formulation of the invention are supplied either separately or mixed together in unit dosage form, for example, as a dry lyophilized powder or water free concentrate in a hermetically sealed container such as a vial, an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The formulations of the invention include bulk drug compositions useful in the manufacture of pharmaceutical compositions (e.g., compositions that are suitable for administration to a subject or patient) which can be used in the preparation of unit dosage forms. In a preferred embodiment, a composition of the invention is a pharmaceutical composition. Such compositions comprise a prophylactically or therapeutically effective amount of one or more prophylactic or therapeutic agents (e.g., an anti-PNAG antibody or other prophylactic or therapeutic agent), and a pharmaceutically acceptable carrier. Preferably, the pharmaceutical compositions are formulated to be suitable for the route of administration to a subject.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the antibody, preferably in purified form, together with a suitable amount of carrier so as to provide the form for proper administration to the patient. The formulation should suit the mode of administration.

Generally, the ingredients of compositions of the invention are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides that the formulation is packaged in a hermetically sealed container such as an ampoule or sachet indicating the quantity of antibody. In one embodiment, the formulation of the invention comprising an antibody is supplied as a dry sterilized lyophilized powder or water free concentrate in a hermetically sealed container and can be reconstituted, e.g., with water or saline to the appropriate concentration for administration to a subject. In certain embodiment, the formulation of the invention comprising the anti-PNAG antibody is supplied as a dry sterile lyophilized powder in a hermetically sealed container at a unit dosage of at least 50 mg, more preferably at least 75 mg, at least 100 mg, at least 150 mg, at least 200 mg, at least 250 mg, at least 300 mg, at least 350 mg, at least 400 mg, at least 450 mg, at least 500 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, at least 2000 mg, at least 2100 mg, at least 2200 mg, at least 2300 mg, at least 2400 mg, at least 2500 mg, at least 2600 mg, at least 2700 mg, at least 2800 mg, at least 2900 mg, or at least 3000 mg of antibody. In a particular embodiment, the anti-PNAG antibody is supplied as a sterile lyophilized dosage unit of 102mg. The lyophilized formulation of the invention comprising an antibody should be stored at between 2 and 8° C. in its original container and the antibody should be administered within 24 hours, including within 12 hours, 6 hours, within 5 hours, within 3 hours, or within 1 hour after being reconstituted. The formulation of the invention comprising antibodies can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### VIII. Combination Therapies

In certain aspects, the present invention includes methods for treating or preventing PNAG-expressing bacterial infection which comprise administering to a subject in need of such treatment an anti-PNAG antibody, or antigen binding fragment thereof, in combination with at least one additional therapeutic agent. Suitable additional therapeutic agents include, without limitation, antibacterial agents (e.g., antibiotics). Suitable anti-bacterial agents include, without limitation, penicillin G, penicillin V, ampicillin, amoxicillin, bacampicillin, cefotaxime, cyclacillin, epicillin, hetacillin, pivampicillin, methicillin, nafcillin, oxacillin, cloxacillin, dicloxacillin, flucloxacillin, carbenicillin, ticarcillin, avlocillin, mezlocillin, piperacillin, amdinocillin, cephalexin, cephradine, cefadoxil, cefaclor, cefazolin, cefuroxime axetil, cefamandole, cefonicid, cefoxitin, cefotaxime, ceftizoxime, cefinenoxine, ceftriaxone, moxalactam, cefotetan, cefoperazone, ceftazidme, imipenem, clavulanate, timentin, sulbactam, neomycin, erythromycin, metronidazole, chloramphenicol, clindamycin, lincomycin, vancomycin, trimethoprim-sulfamethoxazole, aminoglycosides, quinolones, tetracyclines and rifampin (see e.g., Goodman and Gilman's, Pharmacological Basics of Therapeutics, 8th Ed., 1993, McGraw Hill Inc). The amount of the additional therapeutic agent that is administered in the combination therapy can be easily determined using routine methods known and readily available in the art.

### IX. Biomarkers & Genotyping

In certain embodiments, the invention provides methods for treating or preventing a PNAG-expressing bacterial infection by administering to a subject a composition comprising an anti-PNAG antibody, or antigen binding fragment thereof, wherein the level of one or more biomarkers in the subject is modified (e.g., increased, decreased, etc., as the case may be) following administration.

As will be appreciated by a person of ordinary skill in the art, an increase or decrease in a biomarker can be determined by comparing the level of the biomarker measured in the subject at a defined time point after administration of anti-PNAG antibody to the level of the biomarker measured in the subject prior to the administration (i.e., the “baseline measurement”). The defined time point at which the biomarker can be measured can be, e.g., at about 1 hour, 2 hours, 4 hours, 8 hours, 12 hours, 1 day, 2 days, 3 days, 4 days, 5 days, 6 days, 7 days, 8 days, 9 days, 10 days, 14 days, 15 days, 20 days, 21 days, 28 days, 35 days, 40 days, 42 days, 49 days, or more after administration of the anti-PNAG antibody.

According to certain embodiments of the present invention, a subject may exhibit an increase or decrease in the level of one or more biomarkers following administration of an anti-PNAG antibody to the subject. For example, administration of an anti-PNAG antibody, a subject may exhibit an increase or decrease in a biomarker by about 40%, 45%, 50%, 55%, 60%, 65%, 70%, 75%, 80%, 85%, 90%, 95% or more.

The present invention includes methods for determining whether a subject is a suitable patient for whom administration of an anti-PNAG antibody would be beneficial. For example, if an individual, prior to receiving of an anti-PNAG antibody, exhibits a level of a biomarker which signifies potential responsiveness to treatment with of an anti-PNAG antibody, then that individual is identified as a suitable patient for whom administration of an anti-PNAG antibody would be beneficial.

In certain embodiments, the invention provides methods for treating or preventing PNAG-expressing bacterial infection by administering to a patient in need of such treatment a therapeutically effective amount of an anti-PNAG antibody, wherein the presence or absence of one or more Single Nucleotide Polymorphism (SNPs) genotypes in the subject is detected.

The present invention includes methods for determining whether a subject is a suitable patient for whom administration of an anti-PNAG antibody would be beneficial. For example, if an individual, prior to receiving an anti-PNAG antibody, exhibits a SNP genotype which signifies potential responsiveness to treatment with an anti-PNAG antibody, then that individual is identified as a suitable patient for whom administration of an anti-PNAG antibody would be beneficial.

### X. Selection of a Patient Population

In certain embodiments, the methods of the invention include the selection of specific patient populations for treatment. For example, the methods of the invention may include the selection of a subset of patients having a particular PNAG-expressing bacterial infection.

In certain embodiments, the methods of invention include the selection of subjects that have previously been treated for PNAG-expressing bacterial infection using other therapeutic agents, e.g., antibiotics.

In certain embodiments, the methods and compositions described herein are administered to specific patient populations that are refractory to one or more therapeutic agents (other than the anti-PNAG antibodies disclosed herein), e.g., an antibiotic (e.g., vancomycin or methicilin).

### XI. Exemplification

The present invention is further illustrated by the following examples which should not be construed as further limiting. The contents of Sequence Listing, figures and all references, patents and published patent applications cited throughout this application are expressly incorporated herein by reference.

### Example 1

### Production of F598

The anti-PNAG antibody F598 (described in U.S. Pat. No. 7,786,255, which is hereby incorporated by reference in its entirety) was expressed as a human IgG1 from DG44 CHO cells. The antibody was purified using Protein A affinity chromatography and subsequent ion-exchange chromatography. The final antibody preparation conformed to standards appropriate for administration to human subjects.

### Example 2

### Toxicology Studies of F598 in Mice

Two single dose toxicology studies in mice were performed following the recommendations of the FDA as follows:

A. GLP Acute Toxicity Study in CD-1 Mice with Human Monoclonal Antibody F598

Groups of animals were injected via tail vein with vehicle control or a single dose of 1, 10 and 100 mg/kg of F598 followed by a 14 day observation period. At all doses no untoward changes in mortality/morbidity, and body weights were noted. Clinical pathology evaluations noted no changes in hematology, clinical chemistry, and organ weights. No treatment-related histopathologic lesions were observed.

B. Non-GLP Single Dose Toxicity Study of F598 by Intravenous (Bolus) Administration to CD-1 Mice With or Without Administration of Staphylococcus aureus

The systemic toxic potential of F598 alone or followed by administration of S. aureus was assessed following a single intravenous administration to CD-1 mice. Two groups of animals, each comprising of ten male and ten female mice, received F598 (10 mg/kg), with one of these groups also receiving S. aureus (1×10⁵ cfu) approximately 4 hours after F598 administration. A similarly constituted Control group received the vehicle (10 mM Sodium Phosphate, 150 mM NaCl, pH6.5) at the same volume-dose as F598 and also received S. aureus administration. During the study, clinical condition, bodyweight, food consumption, body temperature, hematology, blood chemistry, organ weight and macropathology investigations were undertaken. There were no deaths or effects considered to be clearly related to treatment or of toxicological significance. It was concluded that treatment with F598 alone or followed by administration of S. aureus was not associated with any systemic effects with clear relationship to treatment.

The maximum tolerated and no observed adverse effect level (NOAEL) dose of F598 was not determined from these studies, but was considered to be greater than 100 mg/kg for a single intravenous dose administration.

### Example 3

### Tissue Cross-Reactivity Studies

PNAG, the target antigen for monoclonal antibody F598, is expressed on Staphylococci as well as other bacterial species. However, this specific beta-1-6 linkage between two or more glucosamines, has not, to date, been described to occur in mammalian oligosaccharides. To determine if F598 demonstrated any cross-reactivity with normal tissue, immunohistochemistry studies were performed against a comprehensive FDA screen of frozen normal human tissue specimens.

A comprehensive Tissue Cross-Reactivity (TCR) evaluation was employed for the immunohistochemistry screening of F598 and an isotype control at two different dilutions across triplicate specimens of 33 frozen human tissue types. The tissues evaluated were adrenal, bladder, blood, bone marrow, brain cerebellum, brain cortex, breast, colon, endothelium, eye, fallopian tube, heart, kidney, liver, lung, lymph node, ovary, pancreas, parathyroid, pituitary, placenta, prostate, skeletal muscle, skin, small intestine, spinal cord, spleen, testis, thymus, thyroid, ureter, uterine cervix, and uterine endometrium. In normal human tissues, F598, at concentrations of 0.1 and 0.3 ug/ml, did not bind to any of the 33 human tissues evaluated, with negative staining or only background staining similar to that observed in the IgG isotype control in all tissues tested.

### Example 4

### F598 is Protective in Systemic or Tissue in Preclinical in Vivo Infection Models

The in vivo protective activity of F598 (administered as an intravenous bolus, 4 hours before bacteria challenge) against blood stream infections was determined in a murine septicemia model in immuno-competent mice in which methicillin sensitive S. aureus (strain MN8) was administered intraperitoneally at 1E+06 CFU/mouse. The protective activity of F598 against systemic organ infection was evaluated in skin and respiratory tract infection models, using immuno-competent mice infected with methicillin sensitive S. aureus (strain Smith, administered intramuscularly at 1E+02 CFU/mouse) and S. pneumoniae (strain DSM11869, administered intranasally at 1E+08 CFU/mouse), respectively. Differences in viable bacterial counts between control and drug-treated groups were analyzed by 1-way ANOVA followed by Dunnett's adjustment for multiplicity. For lethal septicemia, differences in survival between control and drug-treated groups were performed using a Log Rank test followed by a Bonferroni-Holm's adjustment for multiplicity

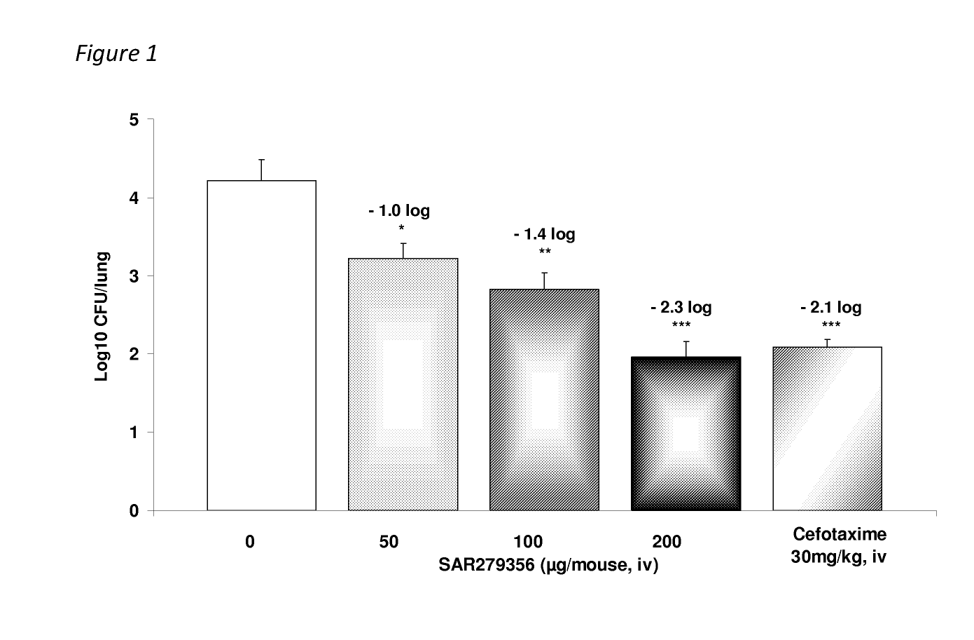

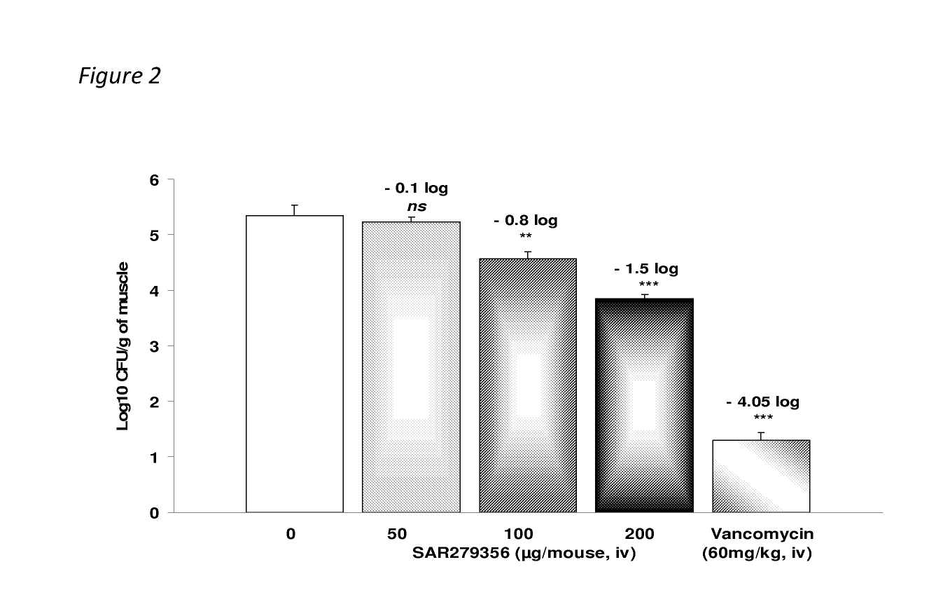

In the S. pneumoniae respiratory tract infection model (FIG. 1) and the S. aureus skin infection model (FIG. 2), administration of F598 4 hours prior to bacterial challenge prevented the development of infection in mice in a dose-dependent manner. A significant prevention effect was observed starting from F598 at 50 or 100 μg/mouse, for the lung and thigh infection model, respectively, with a maximal effect at 200 μg/mouse. In the respiratory tract infection model, the effect of F598 was comparable to the antibiotic Cefotaxime. The protective effect of F598 was also demonstrated in a lethal septicemia model (Table 2), where 300 μg/mouse of F598 prevented 70% of lethality induced by a S. aureus.

TABLE 2
The effect of single doses of F598 on
S. aureus septicemia prevention in mice

| | | | | | | | |
|---|---|---|---|---|---|---|---|
| F598 | 0 | 25 | 50 | 100 | 200 | 300 | Vancomycin |
| (ug/mouse) | | | | | | | (30 mg/kg) |
| % Survival | 0 | 20 | 10 | 30 | 50 | 70 | 100 |
| (96 h post- | | | | | | | |
| infection) | | | | | | | |

### Example 5

### Phase 1 Study of F598

### A) General Study Design

An open-label, dose-escalation Phase 1 study was performed to evaluate the safety, tolerability, pharmacokinetics, pharmacodynamics and immunogenicity of intravenously administered of F598. The study was divided into two parts. Part 1 was a single dose escalation study. Part 2 assessed the effects of a second dose of F598. The dose level and timing of the second dose of F598 was determined by a review of the pharmacokinetic data from Part 1.

Approximately 18 male and female subjects were enrolled in the Phase I single center study to obtain 18 evaluable subjects. This study was designed to assess the safety and tolerability of F598 in healthy volunteers. Approximately 12 subjects were enrolled into Part 1.

A subject was eligible for study participation if he/she meet the following criteria: healthy adult volunteer; at least 18 years of age; normal hematological, hepatic, and renal function was defined by the testing laboratory's normal ranges; non-childbearing potential; or using a medically acceptable contraceptive; males that are not surgically sterile must be practicing a medically acceptable contraceptive regimen; females must have a negative serum pregnancy test; willing to return to the study facility for the post-treatment evaluation; subjects for Part 2 were deemed accessible and able to comply with the prescribed repeat dosing treatment protocol and evaluations; and subject must sign written informed consent and be willing and able to comply with the prescribed treatment protocol and evaluations.

A subject was excluded from the study if he/she meet any of the following criteria: prior therapy with human/humanized antibodies; history of major organ dysfunction; concomitant disease or condition, including laboratory abnormalities, which in the opinion of the investigator could interfere with the conduct of the study or could, put the subject at unacceptable risk; infection or any serious underlying medical condition that would impair the ability of the subject to receive protocol treatment; women who are pregnant or lactating; has an unstable condition or disorder (e.g., psychiatric disorder, a recent history of substance abuse) or otherwise thought to be unreliable or incapable of complying with the requirements of the protocol; or has received any investigational product or device within 30 days before enrollment in this study.

### Part 1—Single Administration of F598

All subjects were observed for a minimum of 50 days to estimate the terminal half-life (T^{1/2}) of a single administration of F598. The starting dose was 1.0 mg/kg/day to be given by intravenous infusion over 30 minutes on Day 1 (see Table 3). The first subject of each dose level was observed for a minimum of 3 days before the remaining subjects in that group could be treated. Dosage escalation occurred only after the final subject at each dose level had been observed for a minimum 3 days and no drug-related toxicity>CTCAE grade 2 had been observed (assessed in accordance with NCI CTCAE, version 4.02).

TABLE 3
Dosage groups for F598 phase 1 study

| | Dose Day 1 | |
|---|---|---|
| Dose Level | (mg/kg/day) | No. Subjects |
| 1 | 1.0 | 4 |
| 2 | 5.0 | 4 |
| 3 | 10.0 | 4 |

### Part 2—Two Administrations of F598

Following review of safety, tolerance, pK, pD and immunogenicity data from Part 1 an additional six subjects received two doses of F598. The dose level and timing of the second dose of F598 was determined by a review of the pharmacokinetic data from Part 1. Dosing and scheduling for Part 2 was based on determination of F598's serum ½-life and protective antibody serum level as measured by the pD opsonic assay. A serum level of 10 ug/ml of F598 was believed to be protective. The protective level of antibody may be longer than the ½-life. Measurement and assessment of those parameters in Part 1 inform selection of an appropriate dose and schedule for Part 2.

F598 for injection was supplied as a clear, colorless liquid in single-use clear glass vials each containing 10 mL F598 solution (prepared at a concentration of 10.2 mg/mL in 20 mM NaPO4, 150mM NaCl, pH 6.5).

All doses of F598 were administered intravenously, via a 30-minute infusion. However, the infusion duration could be extended to up to 2 hours if required by tolerability considerations for a given subject. The infusion solution was prepared fresh on each day of use under aseptic conditions. Prior to preparation of the infusion solution the required number of vials of F598 were brought to room temperature by warming in ambient air for at least 1 hr. The required volume of F598 solution was then removed from the vials, an equivalent volume of normal saline (0.9% NaCl) was be removed from a 100-mL saline infusion bag, and the F598 antibody was added to the infusion bag to provide a final infusion volume of 100 mL. Once prepared, the infusion solution remained at room temperature until the infusion is complete. The infusion commenced within 2 hr following preparation of the infusion solution.

### B) Exemplary Phase 1 Study Results

Healthy adult subjects in 5 cohorts (N=4 per dose) received single doses of F598 (0.86, 4.3, 8.6, 12.9 or 17.2 mg/kg) as a 2 hour intravenous infusion. Pre-infusion and post-infusion samples were obtained over the following 50 days and standard evaluations of the volunteers physical health was carried out for a determination of safety. The presence of F598 in the serum of recipients was evaluated using a human IgG1-specific ELISA to detect binding to PNAG and the PK properties of the mAb analyzed. Pharmacodynamic (PD) assessments were performed using in vitro opsonophagocytic killing (OPK) assay and a flow cytometry based opsonophagocytic assay (OPA).

The opsonophagocytic killing assays were performed by combining isolated human serum with rabbit complement, differentiated HL-60 cells and S.aureus (strain MN8m) and incubating with tumbling at 37° C. for 90 minutes. After incubation, tubes were sonicated for 5 minutes in a water bath sonicator (Fisher Scientific, #FB15050), diluted 100-fold in TSB containing 0.05% Tween80 and plated logarithmically on TSA plates using a spiral plater (IUL, Ensemmenseur Spiral Easy Jet). The serum PNAG antibody titer was determined by quantitating the number of colonies arising on each plate. In the assay, S.aureus (strain MN8m) were prepared by growing for 16 hours at 37° C. in TSB +2% NaCl, with shaking at 240rpm. When the bacterial culture reached about OD₆₀₀ 0.4, the bacteria were diluted 200× in HBSS/0.1% gelatin. Baby rabbit complement (#31061-batch #21832L, PelFreez Biologicals) was pre-adsorbed on protein G-sepharose (Sigma # P3296-5ML) for 1 hour at 4° C. Prior to use, HL-60 cells were permitted to differentiate for 3-4 days in RPMI1640/10% FBS/Glutamax 1×/0.8% DMF at a cell concentration of 4×10⁵ cells/ml.

The opsonophagocytic assays were performed by combining isolated human serum with rabbit complement, differentiated HL-60 cells and fluorescently labeled S.aureus (strain MN8m) and incubating with rotation at 37° C. for 30 minutes. The serum PNAG antibody titer was determined by quantitating the amount of bacteria phagocytosed by the HL60 cells using a FACS machine. In the assay 5,6 Carboxyfluorescein succinimidyl ester-labelled S. aureus strain MN8 (Molecular Probes # C-1311) was used. Differentiated HL60 cells were pre-washed twice in buffer (the second wash step being performed for at least 20 minutes) and diluted to a concentration of 2.5×10⁴ cells /40 μl in assay buffer.

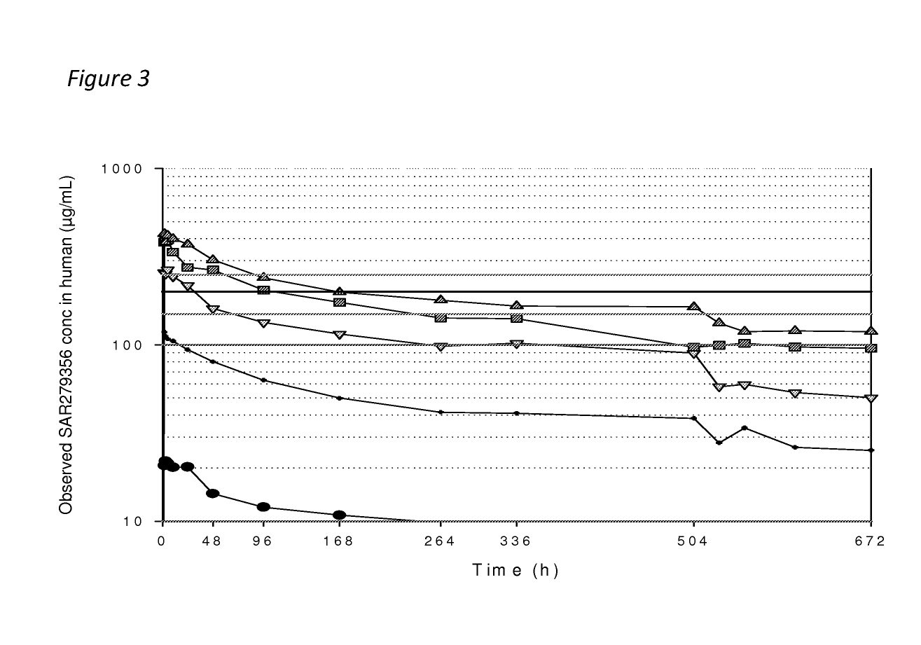

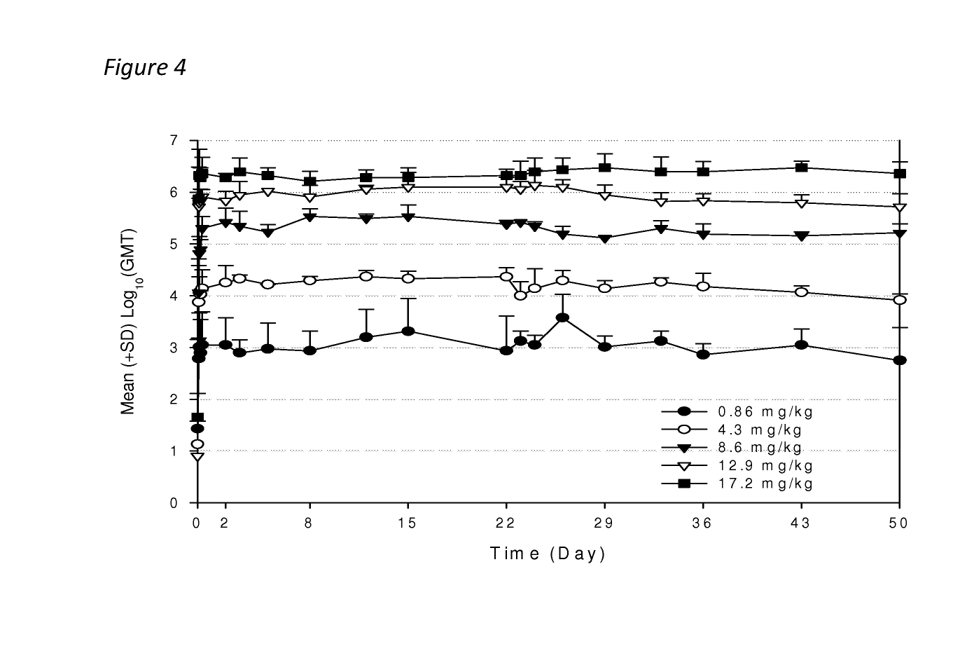

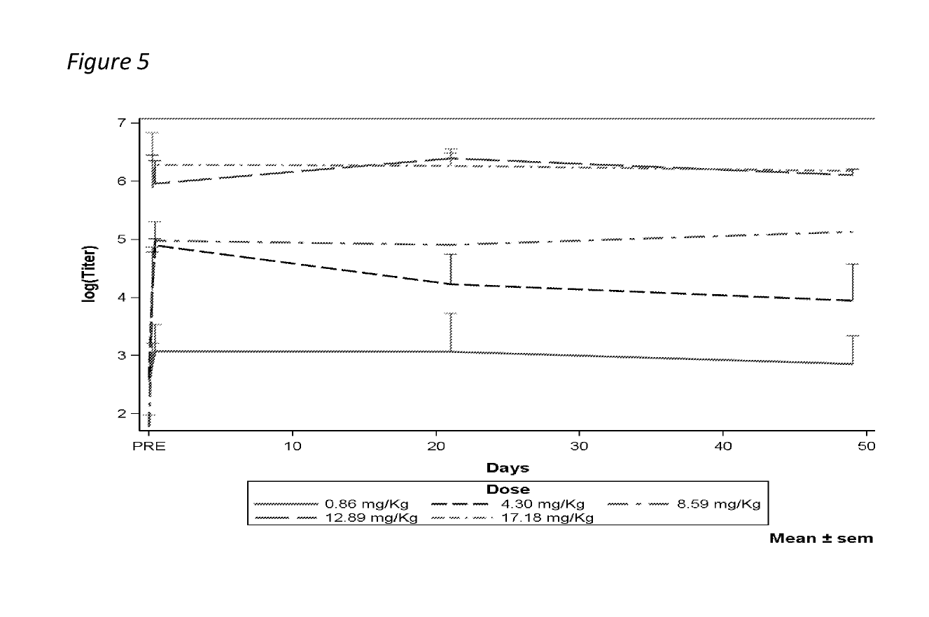

The results set forth in Table 4 and FIGS. 3, 4, and 5 show that F598 exhibited a long terminal elimination half-life (˜20-30 days), a low systemic clearance of 0.0699 to 0.0851 mL/h/kg and a low apparent volume of distribution of 53.4 to 67.0 mL/kg. A dose-dependent increase in opsonophagocytotic activity was observed. For each dose, this activity was maximal starting from the first time point post-dose (4 h) and did not decrease over time (up to day 50).

TABLE 4
Pharmacokinetics of F589 in human subjects

| Dose | T_{1/2} (h) | Cl (mL/hrkg) | Vss (mL/kg) |
|---|---|---|---|
| 0.86 mg/kg | 656 ± 277 | 0.0717 ± 0.0235 | 62.2 ± 6.40 |
| 4.3 mg/kg | 485 ± 30.4 | 0.0851 ± 0.00651 | 61.3 ± 8.13 |
| 8.6 mg/kg | 468 ± 41.0 | 0.0780 ± 0.0111 | 53.4 ± 3.23 |
| 12.9 mg/kg | 622 ± 176 | 0.0699 ± 0.0142 | 59.9 ± 10.3 |
| 17.2 mg/kg | 714 ± 174 | 0.0709 ± 0.0115 | 67.0 ± 4.35 |

### Example 6

### Phase 2a Study of F598

### A) Study Design

A randomized, double-blind, placebo-controlled Phase 2a study was performed to assess the pharmacokinetics, pharmacodynamics, and safety of a single intravenous dose of F598 in patients hospitalized in the intensive care unit and on mechanical ventilation. Six patients were treated in this study. A subject was eligible for study participation if he/she was hospitalized in the intensive care unit and on mechanical ventilation.

F598 was formulated as a liquid at a concentration of 17.0 mg/mL. The drug was administered by intravenous infusion in 250 mL of normal saline (0.9% NaCl) over 2 hours for both dose regimens. Patients were given a single dose of the drug at a concentration of either 8.6 mg/kg or 12.9 mg/kg.

The placebo was a saline solution (0.9% NaCl), which was administered by intravenous infusion in 250 mL over 2 hours.

Patients were observed for a total of 91 days. Patients were screened 1 day prior to dosing, given one intravenous injection of F598 on day 1, with a follow-up visit in 90 days.

The following pharmacokinetic (PK) parameters were calculated for F598 using a non-compartmental method: serum concentration at the end of infusion (Cₑₒᵢ), area under the serum concentration versus time curve from time 0 to the real time tlast, time of last measured serum concentration (tₗₐₛₜ) area under the serum concentration versus time curve extrapolated to infinity (AUC), terminal half-life (t_{1/2z}), total body clearance of a drug from the serum (CL), and volume of distribution at steady state (V_{SS}).

The efficacy/pharmacodynamics endpoints were as follows. There was no primary endpoint. The secondary endpoints were: pharmacodynamic: opsonic titers over time; immunogenicity: human anti-human antibodies (HAHA); exploratory efficacy: documented infections caused by PNAG expression pathogens up to Day 28; and exploratory efficacy: duration of mechanical ventilation, ICU stay, hospital stay, 28-day and 90-day all-cause mortality.

The safety endpoints were: acute infusion reactions, treatment-emergent adverse events (TEAE) up to Day 90 and standard hematology and blood chemistry, and blood cultures and endotracheal aspirates (ETA) cultures (for patients under mechanical ventilation).

The pharmacokinetic/pharmacodynamics sampling times and bioanalytical methods were as follows:

PK:
Sampling times: blood samples were collected at predose, 1, 2 hours (end of infusion) and 8 hours on Day 1, then on Days 2, 5, 7, 14, 21, 28, 56, 70 and 90 postdose.
Bioanalytical methods: Serum concentrations of F598 were determined by a validated enzyme-linked immmunosorbent assay (ELISA) with a lower limit of quantification (LLOQ) of 0.0858 μg/mL

Human anti-F598 antibodies (HAHA):
Sampling times: blood samples were collected at predose, then on Days 14, 28, 56 and 90 postdose.
Bioanalytical methods: Serum HAHA was detected by a validated electro chemiluminescence (ECL) immunoassay.

PD:
Sampling times: Opsonophagocytic test (OPA): Day 1: Predose, 2 hours (end of infusion), Day 2, 7, 14, 28, 56, 90; Killing test (OPK) samplings: Day 1: Predose, 2 hours (end of infusion), Day 14, Day 28, Day 90.
Bioanalytical methods OPA: Matrix: serum/Analytical technique: OPA/Lower limit of quantification: NA/Assay volume: NA/Site of bioanalysis: Flowapps/Method of reference: FA-440.
Bioanalytical methods OPK: Matrix: serum/Analytical technique: OPA/Lower limit of quantification: NA/Assay volume: NA/Site of bioanalysis: Flowapps/Method of reference: FA-445.

The statistical methods were as follows. The primary analyses were summaries of PK variables (Cₑₒᵢ, AUCₗₐₛₜ, AUC, CL, V_{SS}, t_{1/2z} and tₗₐₛₜ) using descriptive statistics by dose. The analyses were based on the PK population consisting of all randomized patients with a least one PK-evaluable data point. The safety population was defined as all patients who were randomized and administered by infusion the study medication regardless of whether the infusion was completed or not. Safety data were summarized by the actual treatment or dose received, unless indicated otherwise. Adverse event incidence tables were presented by actual treatment or dose received, system-organ-class (SOC) and preferred term (PT).

### B) Results

The results of the Phase 2a study were as follows. Six patients received the F598 infusion; 2 received the placebo, 2 received 8.6 mg/kg F598, and 2 received 12.9 mg/kg F598. Only the patients who were administered the F598 infusion were considered for PK, safety, and other exploratory assessments.

The F598 PK parameters after single IV doses are summarized in Table 5 below.

TABLE 5
Individual values of F598 pharmacokinetic parameters

| Parameter | 8.6 mg/kg | 12.9 mg/kg |
|---|---|---|
| Cₑₒᵢ (μg/mL) | 147, NC ^{b} | 268, 184 |
| tₗₐₛₜ ^{a} (h) | 643-1920 | 312-1410 |
| AUCₗₐₛₜ (μg · h/mL) | 50600, 56800 | 27900, 67000 |
| AUC (μg · h/mL) | 69200, 71900 | 78000, NC ^{c} |
| T_{1/2z} (h) | 353, 632 | 275, 585 |
| CL (mL/h/kg) | 0.12, 0.12 | 0.17, NC ^{c} |
| Vₛₛ (mL/kg) | 61.0, 96.8 | 107, NC ^{c} |

^{a} range; PK samples were not collected beyond time point corresponding to tₗₐₛₜ whereas it was planned to collect them up to Day 90 (T2160 h);
^{b} Not calculated, due to no sampling at the end of infusion in one subject
^{c} Not calculated, due to AUC extrapolation >30% in one subject
All values are expressed to three significant figures

We claim:

1. A method for treating or preventing a poly-N-acetyl glucosamine (PNAG)-expressing bacterial infection in a subject comprising administering to the subject a therapeutically effective amount of an anti-PNAG antibody, wherein the antibody is administered at a dose of about 0.5 to about 20 mg/kg.

2. The method of claim 1, wherein the antibody is administered at a dose of about 10, about 15, about 17 or about 20 mg/kg.

3. The method of any one of the preceding claims, wherein the administered dose achieves a serum level of anti-PNAG antibody of at least 10 μg/ml.

4. The method of any one of the preceding claims, wherein the opsonic activity of the subject's serum remains essentially constant for at least 50 days after administration of the antibody.

5. The method of any one of the preceding claims, wherein the antibody has a serum half-life of at least 25 days.

6. The method of any one of the previous claims, wherein the antibody is administered as a single dose.

7. The method of any one of the previous claims, wherein the antibody is administered in multiple doses.

8. The method of claim 7, wherein the dosing and scheduling of at least one dose is based upon a determination of the antibody's serum half-life in the subject and/or the in vitro opsonic activity of the subject's serum against PNAG-expressing bacteria.

9. The method of any one of the preceding claims, wherein the antibody is administered by intravenous infusion.

10. The method of claim 9, wherein the intravenous infusion is administered over about 30 to about 120 minutes.

11. The method of claim 9, wherein the intravenous infusion volume is about 100 ml.

12. The method of any one of the preceding claims, wherein the subject has a PNAG-expressing bacterial infection.

13. The method of any one of the preceding claims, wherein the subject is at risk of developing a PNAG-expressing bacterial infection.

14. The method of claim 13, wherein the infection is a lung infection, joint infection, endocardial infection, skin infection, soft tissue infection, or septicemia.

15. The method of any one of the preceding claims, wherein the antibody is administered before, after or during a medical procedure.

16. The method of claims 15, wherein the medical procedure is the installation of a surgical implant in the subject.

17. The method of claim 16, wherein the surgical implant is a stent, catheter, cannula, prosthesis, or pace-maker.

18. The method of any one of the preceding claims, wherein the subject is a human.

19. The method of any one of the preceding claims, wherein the PNAG-expressing bacterial infection comprises Staphylococcus.

20. The method of claim 19, wherein the Staphylococcus is S. epidermidis or S. aureus.

21. The method of claim 20, wherein the S. aureus is Methicillin-resistant S. aureus.

22. The method of any one of the preceding claims, further comprising determining the effective serum titer of the administered antibody using an in vitro opsonophagocytosis assay.

23. The method of any one of the preceding claims, wherein the antibody is in a formulation comprising:
i) 10.2 mg/ml of anti-PNAG antibody;
ii) 20 mM NaPO₄; and
iii) 150 mM NaCl,
wherein the pH of the formulation is 6.5.

24. The method of any one of the preceding claims, wherein the antibody is a human antibody.

25. The method of any one of the preceding claims, wherein the antibody comprises a heavy chain variable region comprising the HCDR1, HCDR2, and HCDR3 amino acid sequences set forth in SEQ ID NOs: 1, 2, and 3, respectively.

26. The method of any one of the preceding claims, wherein the antibody comprises a light chain variable region comprising the LCDR1, LCDR2, and LCDR3 amino acid sequences set forth in SEQ ID NOs: 4, 5, and 6, respectively.

27. The method of any one of the preceding claims, wherein the antibody comprises a heavy chain variable region comprising the HCDR1, HCDR2, and HCDR3 amino acid sequences set forth in SEQ ID NOs: 1, 2, and 3, respectively, and a light chain variable region comprising the LCDR1, LCDR2, and LCDR3 amino acid sequences set forth in SEQ ID NOs: 4, 5, and 6, respectively.

28. The method of any one of the preceding claims, wherein the antibody comprises a heavy chain variable region comprising the amino acid sequence set forth in SEQ ID NO: 7.

29. The method of any one of the preceding claims, wherein the antibody comprises a light chain variable region comprising the amino acid sequence set forth in SEQ ID NO: 8.

30. The method of any one of the preceding claims, wherein the antibody comprises a heavy chain variable region comprising the amino acid sequence set forth in SEQ ID NO: 7, and a light chain variable region comprising the amino acid sequence set forth in SEQ ID NO: 8.
